(19) United States
(12) Patent Application Publication
(10) Pub. No.: US 2016/0367142 A1
(43) Pub. Date: Dec. 22, 2016

(54) MEDICAL IMAGING APPARATUS IN A MEDICAL IMAGING SYSTEM, AND SYSTEM CONTROL COMPUTER FOR OPERATION THEREOF DURING A MEDICAL IMAGING EXAMINATION

(71) Applicant: Siemens Healthcare GmbH, Erlangen (DE)

(72) Inventors: Donald Hardie, Moehrendorf (DE); Anja Kuerten, Obermichelbach (DE); Stephan Nufer, Erlangen (DE); Thorsten Speckner, Erlangen (DE); Jens Thoene, Nuernberg (DE)

(73) Assignee: Siemens Healthcare GmbH, Erlangen (DE)

(21) Appl. No.: 15/185,622

(22) Filed: Jun. 17, 2016

(30) Foreign Application Priority Data
Jun. 17, 2015 (DE) 102015211140.7

(51) Int. Cl. A61B 5/00; A61B 6/00; A61B 5/055; A61B 6/03; G05B 15/02; G06F 19/00

(52) U.S. Cl. CPC A61B 5/0046; G05B 15/02; G06F 19/3406; A61B 5/055; A61B 6/032; A61B 6/037; A61B 5/7475; A61B 6/54; A61B 5/742

(57) ABSTRACT

In a method for operating a medical imaging apparatus of a medical imaging system during a medical imaging examination, wherein the medical imaging system includes the medical imaging apparatus, which has a scanner for the acquisition of medical image data and which is arranged within an examination area, and a system-control unit, which has an operation-control module and which is arranged within a control area, the medical imaging system comprises a user interface, which is arranged within the examination area, wherein an output of information and/or suggestions and/or an input of parameters and/or information for operating the medical imaging apparatus during an implementation of the medical imaging examination ensues via the user interface.

### BACKGROUND OF THE INVENTION

Field of the Invention

The present invention concerns a method for operating a medical imaging apparatus of a medical imaging system during a medical imaging examination, wherein the medical imaging system includes the medical imaging apparatus, which has a scanner for the acquisition of medical image data and that is situated in an examination area, and a system control computer, which has an operation-control module, and that is situated in a control area. The present invention further concerns a medical imaging system with a medical imaging apparatus that has a scanner for the acquisition of medical image data and that is arranged within an examination area, and a system-control unit that has an operation-control module and that is arranged within a control area.

Description of the Prior Art

During a medical imaging examination, it is conventional to operate a medical imaging apparatus, such as a magnetic resonance apparatus, almost exclusively from a control area that is separate from an examination area in which the medical imaging apparatus is situated and in which the medical imaging examinations take place. Conventionally, operation of a medical imaging apparatus during the medical imaging examination has not been possible within the examination area. The operation of the medical imaging apparatus during the medical imaging examination includes supervision and/or monitoring and/or control of the medical imaging examination, for example, the entry of examination-related parameters into the control computer.

This is particularly problematic with patients who require an increased level of care during the medical imaging examination, because the medical operator has to remain inside the control area during the medical imaging examination to operate the medical imaging apparatus. With patients requiring a high level of care, the medical operator therefore has to move between the control area and the examination area. Patients requiring a high level of care may be, for example, children, emergency patients, nervous patients, patients with a claustrophobic disposition, etc.

### SUMMARY OF THE INVENTION

The present invention addresses the problem of enabling a medical operator to operate a medical imaging apparatus from an examination area during the medical imaging examination.

The invention encompasses a method for operating a medical imaging apparatus of a medical imaging system during a medical imaging examination, wherein the medical imaging system includes the medical imaging apparatus, which includes a scanner for the acquisition of medical image data that is arranged within an examination area, and a system control computer, which has an operation-control module and that is arranged within a control area.

The medical imaging system has a user interface that is situated within the examination area, with an output of information and/or suggestions and/or an input of parameters and/or information for operating the medical imaging apparatus ensuing via the user interface while carrying out the medical imaging examination.

The medical imaging system may be any medical imaging system that seems practical to to those skilled in the art, such as a computed tomography system, a PET System (Positron Emission Tomography System) and so on. It is particularly advantageous, however, when the medical imaging system is to be a magnetic resonance system, because this involves a relatively long examination period, in which the patient has to lie inside a confined patient-accommodating area for magnetic resonance examinations, if possible without moving at all.

The user interface preferably includes an input interface and an output interface. The input interface and the output interface are designed as one component and/or together in one component. The operating module preferably includes software and/or computer programs that are designed to operate the medical imaging apparatus while carrying out the medical imaging examination from the examination area. The software and/or computer programs are stored in a memory of the system control unit, in particular of the operating module, and are carried out by a processor of the operating module.

As used herein, an examination area means an area in which the medical imaging apparatus, in particular the scanner of the medical imaging apparatus, is situated. Preferably, the examination area is shielded both outwardly and inwardly against interfering influences, which can both affect a medical imaging examination and may also be caused by the medical imaging apparatus itself. For example, if the medical imaging apparatus is a magnetic resonance apparatus, the examination area is shielded both with regard to magnetic fields and from electromagnetic radiation, in particular from radio-frequency radiation. If the medical imaging apparatus is a computed tomography apparatus, the examination area can be shielded from electromagnetic radiation. All the units and modules to control the medical imaging apparatus are preferably arranged in the control area. The control area is shielded from the examination area with regard to magnetic fields and against electromagnetic radiation, in particular radio-frequency radiation.

The invention allows a medical operator, for instance a managing physician or a supervising technician, to operate the medical imaging apparatus from an examination area while carrying out the medical imaging examination. Operating may include here controlling the running of the medical imaging examination and/or also supervising and/or monitoring the medical imaging examination. In particular, while doing this, the medical operator can remain beside the patient inside the examination area during the medical imaging examination and operate the medical imaging apparatus at the same time, for example, to enter at least one examination parameter and/or to select an examination protocol, etc.

The present invention also makes it possible to support the medical operator, for example a physician who is managing the examination, during an interventional examination of the patient, because while doing so the medical operator can implement control commands directly within the examination area via the user interface. The interventional examination may include a biopsy, for example. The interventional examination may also include insertion of a catheter and/or a probe, for example, a heat probe and/or a cold probe in the case of liver cancer in the patient. Via the user interface, individual measurement steps in the medical imaging examination can be adjusted to the interventional procedure. In addition, via the user interface, in particular via a display unit in the user interface, the medical operator can have additional information displayed, which the medical operator can use to adjust and/or determine a next step in the procedure. This allows continuous monitoring and control of the interventional procedure directly at the site of the interventional examination, that is, inside the examination area.

The invention also allows rapid intervention during the medical imaging examination where there is a patient problem, for example, where the patient is unwell, without the operator having to leave the area to operate, such as to monitor and/or control, the medical imaging apparatus, as the operation conventionally had to. This also allows a particularly time-saving implementation of medical imaging examinations, particularly with patients requiring a high level of care. Preferably, a complete operating procedure, such as control and/or monitoring, of the medical imaging apparatus, can ensue for a medical operator while implementing the medical imaging examination from the examination area.

In an embodiment, an output of information and/or suggestions and/or an input of parameters and/or information for operating the medical imaging apparatus while carrying out the medical imaging examination ensues via the user interface. In this way, after preparation of the medical imaging examination, the medical operator can remain in the examination area and start the medical imaging examination and does not have to go to the control area after completing the preparation. This may likewise lead to a particularly time-saving implementation of the medical imaging examination.

In a further embodiment of the invention, suggestions and/or instructions for various medical imaging examinations are stored in a memory of the system-control unit, and are retrieved for the respective medical imaging examination on the user interface. This allows a simple, guided implementation of medical imaging examinations and safe operation of the medical imaging apparatus even for an inexperienced medical operator. In this case, it is also possible to carry out, in a simple manner that is as error-free as possible, medical imaging examinations that are not included within the routine activities of the medical operator. Furthermore, as a result, it is also possible to carry out medical imaging examinations in regions and/or countries that have, for example a low level of training for a medical operator, since the medical operator can be guided safely through the medical imaging examination by means of the downloadable instructions and support.

In another embodiment, control of the user interface ensues by operation of the operating module. This allows safe control of the user interface, protected against malfunction, since the operation control module is arranged inside the control area.

In another embodiment, monitoring of the medical imaging examination ensues on an operating console, the operating console being arranged outside the examination area. In this way, advantageous support of an operator carrying out the medical imaging examination can be achieved by a user, familiar with the specialist area and/or with the medical imaging examination logging into the system via the operating console so as to provide additional support. The operating console can be arranged inside the control area or even in a further area of the hospital and/or building in which the medical imaging system is arranged. The operating console also can be arranged in a different building and/or in a different country.

Furthermore, the invention encompasses a medical imaging system having a medical imaging apparatus that has a scanner for the acquisition of medical image data that is arranged within an examination area, and a system-control unit, which has an operation-control module, that is arranged within a control area.

The medical imaging system has a user interface for operating the medical imaging apparatus, the user interface being arranged within the examination area, operation of the medical imaging apparatus ensuing via the user interface during implementation of the medical imaging examination.

The invention allows a medical operator, for instance a managing physician or a radiology technician, etc., to operate the medical imaging apparatus from an examination area during implementation of the medical imaging examination. While doing this, the medical operator can remain beside the patient inside the examination area during the medical imaging examination and in addition operate the medical imaging apparatus at the same time, by, for example, entering at least one examination parameter and/or making a selection of an examination protocol.

In addition, the invention also allows rapid intervention during the medical imaging examination where there is a patient problem, for example, where the patient is unwell, without having to leave an area to operate, in particular to monitor and/or control, the medical imaging apparatus, as was previously necessary. Preferably, a complete operating procedure, in particular control and/or monitoring, of the medical imaging apparatus can consequently ensue for a medical operator during implementation of the medical imaging examination from the examination area.

The present invention also makes it possible to support the medical operator, for example a physician who is managing the examination, during an interventional examination of the patient, since here the medical operator can implement control commands directly within the examination area by means of the user interface. The interventional examination may include a biopsy, for example. In addition, the interventional examination may also include insertion of a catheter and/or a probe, for example a heat probe and/or a cold probe, in the case of liver cancer in the patient. Via the user interface, individual measurement steps in the medical imaging examination can be adjusted advantageously to the interventional procedure. In addition, via the user interface, in particular via a display unit of the user interface, the medical operator can have additional information displayed, with which the medical operator can adjust and/or determine a next step in the procedure. This allows continuous monitoring and control of the interventional procedure directly at the site of the interventional examination, that is, inside the examination area.

The advantages of the medical imaging system according to the invention essentially correspond with the advantages of the method according to the invention for carrying out a medical imaging examination by means of a medical imaging system, which were described in detail above. Features, advantages or alternative embodiments can likewise be applied to the other aspect of the invention.

In a further embodiment, the user interface includes a touch display. In this way, display of information and moreover input of parameters and/or positioning data for a medical operator can ensue in a particularly simple manner during operation of the medical imaging apparatus.

As used herein, a touch display means a touch-sensitive screen on which an input of parameters can ensue by touching partial areas of a screen, in particular partial areas of an image displayed on the screen, for example to control a program run.

In a further variant of the invention, the user interface is fixedly arranged on the medical imaging apparatus. Due to the arrangement of the user interface in a fixed location, information is constantly displayed in the same place, such that good visibility is provided for the medical operator. Moreover, the user interface is in particular arranged inside the examination area so that it cannot be lost. The user interface is preferably arranged on a housing that surrounds the detector unit and/or on a patient-accommodating device of the medical imaging apparatus.

In another embodiment, the user interface is removably arranged on the medical imaging apparatus, as a result of which a flexible arrangement of the user interface can be achieved. For example, the user interface can be arranged here within a working area of the medical operator, such that direct accessibility of the user interface is constantly provided in this way.

In a further variant of the invention, control of the user interface is achieved by the operating module. This allows safe control of the user interface, protected against malfunction, since the operation control module is arranged inside the control area.

In a further embodiment, the medical imaging system has an operating console which is arranged outside the examination area. In this way, advantageous support of the operator carrying out the medical imaging examination can be achieved, in that, by means of the operating console, a user familiar with the specialist area can log into the system and provide additional support. The operating console can be arranged inside the control area or even in a further area of the hospital and/or building in which the medical imaging system is arranged. The operating console also can be arranged in a different building and/or in a different country.

The invention further encompasses a non-transitory, computer-readable data storage medium encoded with program code, the storage medium being loadable directly into a memory of a programmable operating module of a medical imaging system. The program code causes the method as described above to be implemented when the program code runs in the operation control module of the medical imaging system. The computer program may require peripheral means, such as libraries and auxiliary functions, in order to implement the relevant embodiments of the method. The computer program may include software with a source code that still has to be compiled and linked up or just has to be interpreted, or an executable software code that just has to be loaded into an appropriate computation unit to run the program.

### BRIEF DESCRIPTION OF THE DRAWINGS

FIG. 1 is a schematically illustration of a medical imaging system according to the invention.

FIG. 2 is a flowchart of the method according to the invention for operating the medical imaging system during a medical imaging examination.

### DESCRIPTION OF THE PREFERRED EMBODIMENTS

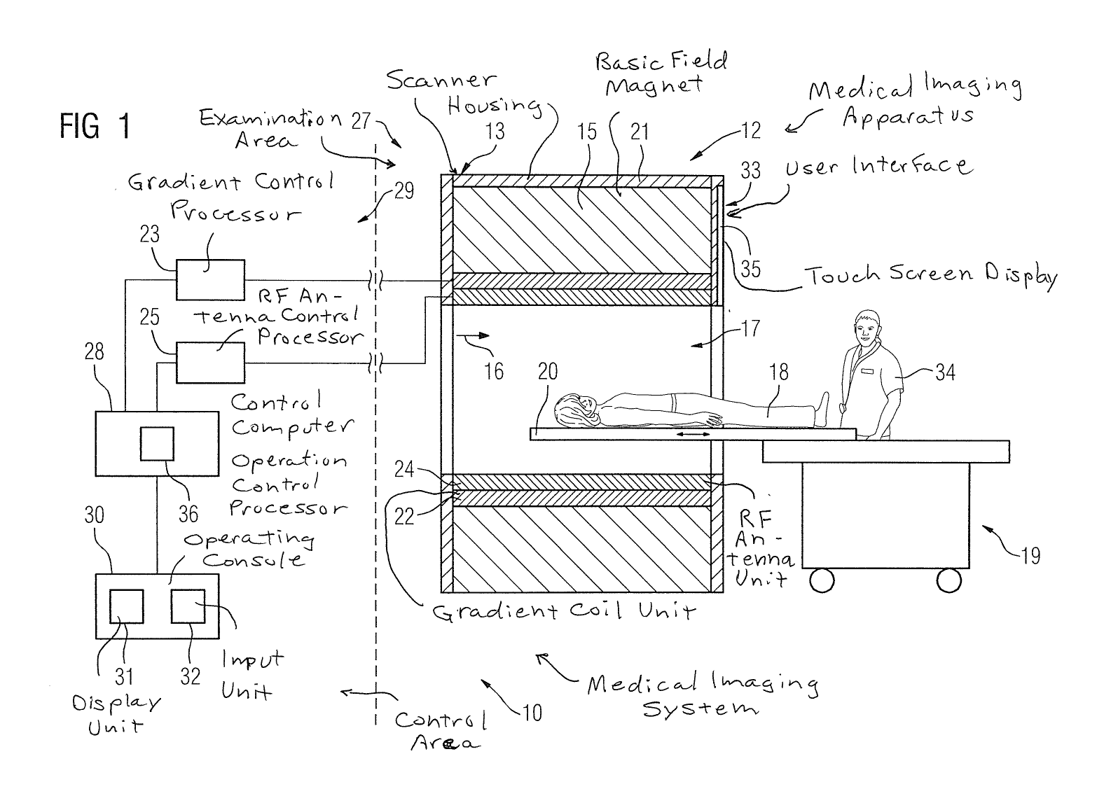

FIG. 1 schematically shows a medical imaging system 10. In the present embodiment, the medical imaging system 10 is a magnetic resonance system. The design of the medical imaging system 10 is not restricted to a magnetic resonance system. The medical imaging system 10 may be any medical imaging system 10 that appears practical to those skilled in the art, such as a computed tomography system, a positron emission tomography system, etc.

The medical imaging system 10 includes a medical imaging apparatus 12, in this case a magnetic resonance apparatus with a scanner 13 that has a superconducting basic field magnet 15 to generate a strong and constant basic magnetic field 16. The scanner 13 has a patient-accommodating area 17 to accommodate a patient 18. In the present embodiment, the patient-accommodating area 17 is cylindrical in design and is encompassed circumferentially by the scanner 13. Another design of the patient-accommodating area 17 that deviates therefrom can be used. The patient 18 can be moved by a patient-positioning apparatus 19 of the medical imaging apparatus 12 into the patient-accommodating area 17. To this end, the patient-positioning apparatus 19 has a patient table 20 that is moveably configured within the patient-accommodating area 17.

The scanner 13 is surrounded by a housing 21.

The scanner 13 further has a gradient coil unit 22 to apply magnetic field gradients that are used for spatial coding during imaging. The gradient coil unit 22 is controlled by a gradient control processor 23. The scanner 13 further has a radio-frequency antenna unit 24 to excite nuclear spins in the patient 18 so as to cause the spins to deviate from the polarization that is established in the basic magnetic field 16 generated by the basic field magnet 15. The radio-frequency antenna unit 24 is controlled by a radio-frequency antenna control processor 25 so as to radiate radio-frequency magnetic resonance sequences into the patient-accommodating area 17 of the scanner 13.

The medical imaging apparatus 12, in particular the magnetic resonance apparatus, is arranged inside an examination area 27. The examination area 27 is shielded with respect to electromagnetic radiation and/or with respect to dispersion of a magnetic field.

For controlling the basic field magnet 15, the gradient control processor 23, and the radio-frequency antenna control processor 25, the imaging apparatus 12 has a system control computer 28. The system control computer 28 is arranged within the control area 29, which is configured separate from the examination area 27, usually with a physical barrier therebetween represented by the dashed line. In particular, the control area 29 is decoupled from the examination area 27 with respect to dispersion of a magnetic field and/or of electromagnetic radiation.

The system control computer 28 centrally controls the magnetic resonance apparatus forming the medical imaging apparatus 12, such as to run a predetermined image-generating gradient echo sequence. In addition, the system control processor 28 includes an evaluation processor 36, which is not described in further detail, to evaluate medical image data that are acquired during a magnetic resonance examination. Furthermore, the magnetic resonance unit 11 has an operating console 30, which is connected to the system control computer 28. The operating console 30 is arranged outside the examination area 27. Moreover, the operating console 30 is arranged together with the system control computer 28 inside the control area 29 and comprises a display unit 31 and an input unit 32.

The medical imaging system 10 further has a user interface 33, which is designed for presentation of information and/or suggestions to a medical operator 34 and/or an input of parameters and/or to receive information by the medical operator 34 for operating the medical imaging apparatus 12, in particular the magnetic resonance apparatus, during an implementation of the medical imaging examination, in particular of a magnetic resonance examination. The user interface 33 has a display unit and an input unit. In the present embodiment, the display unit and the input unit are designed in one piece as a touch display 35.

The user interface 33, in particular the touch display 35, is arranged inside the examination area 27 together with the magnetic resonance apparatus forming the medical imaging apparatus 12. The touch display 35 is arranged on the magnetic resonance apparatus 12. In the present embodiment, the touch display 35 is arranged on the housing 21 of the scanner 13. Basically, however, an arrangement of the touch display 35 on the patient-accommodating apparatus 19, for example, and/or on further components of the medical imaging apparatus 12 and/or inside the examination area 27 is completely conceivable.

The touch display 35 is arranged on a front face of the housing unit 21. In addition, the touch display 35 is removably arranged on the housing 21 such that the touch display 35 can be removed by the medical operator 34 in the event of actions that only allow a poor view onto the front of the housing unit 21 and/or are a fairly considerable distance from the front of the housing unit 21 and can be arranged in an angle of view and/or a working area of the medical operator 34.

In addition, in an alternative variant of the invention, a fixed arrangement of the touch display 35 is also conceivable in which the touch display 35 cannot be removed from the housing unit 21.

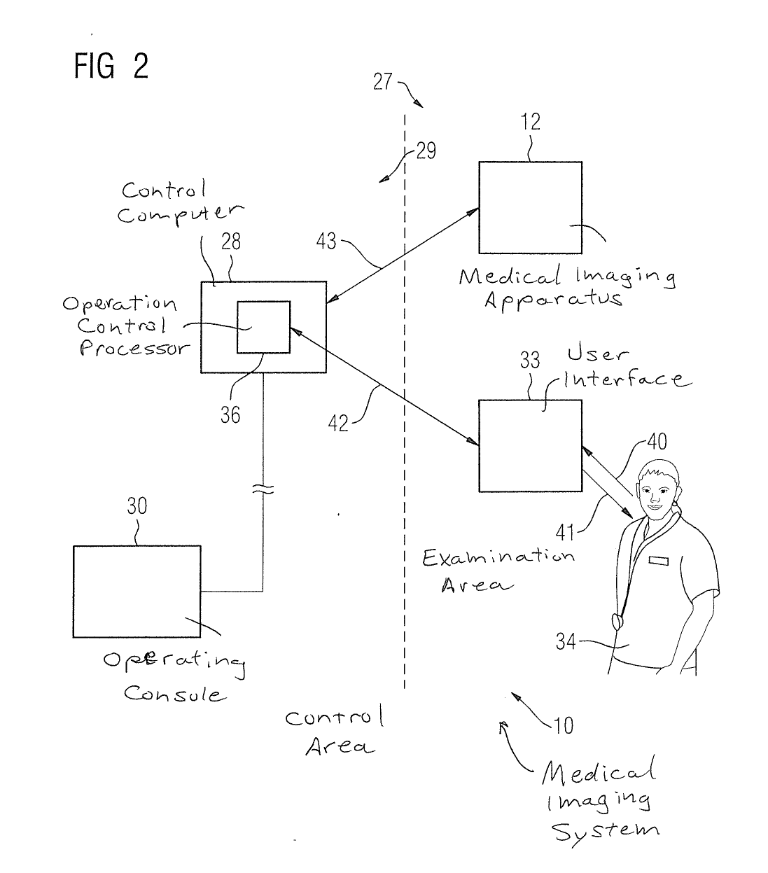

Control of the touch display 35 is achieved by means of the system control computer 28. For this purpose, the system control computer 28 has an operation control processor 36, which is designed to control the user interface 33, in particular the touch display 35. The operation control processor 36 has appropriate control software and/or computer programs, which are stored in a memory unit that is not described in greater detail of the operation control processor 36. The control software and/or the computer programs are run by a processor of the operation control processor 36. For a data exchange, the operation control processor 36 is connected with the user interface 33, as can be seen in FIG. 2.

FIG. 2 shows a method according to the invention for operating the medical imaging apparatus 12 of the medical imaging system 10 during a medical imaging examination. The method is performed by the system control computer 28, in particular by the operation control processor 36 of the system control computer 28, together with the user interface 33. To this end, the operation control processor 36 has appropriate control software and/or computer programs, which are stored in a memory unit of the operation control processor 36 that is not described in greater detail. The control software and/or the computer programs are run by the operation control processor 36.

By means of the user interface 33, in particular of the touch display 35, there follows input 40 of parameters, such as measurement parameters and/or control parameters, for example, and/or selection of an examination protocol and so on, and/or of further information, for example, a confirmation of a successful implementation of a working step, for the medical imaging examination from the examination area 27. An output 41 to the medical operator 34 of information and/or data, in particular a display and/or retrieval of additional information and/or suggestions, for example individual steps of instructions for specific examinations, can likewise ensue by means of the touch display 35. In addition, additional suggestions or information can also be displayed to the medical operator 34 during the medical imaging examination by means of the touch display 35. In this scenario, the medical operator 34 can directly retrieve or input the required information and/or parameters directly from the examination area to control the medical imaging apparatus 12.

Furthermore, it is also conceivable for an output 41 of information and/or suggestions and/or an input 40 of parameters and/or information to ensue during a preparation of the medical imaging examination directly inside the examination area 27 by means of the user interface 33, in particular by means of the touch display 35. Such information and/or suggestions that are issued to the medical operator 34 by means of the touch display 35 can be, for example, an instruction and/or the next steps in the procedure to prepare the patient 18 for the impending imaging examination. Moreover, such information and/or suggestions can be warnings regarding the dangers when positioning the patient 18 and/or additional units, such as an ECG unit, for example, and/or a local radio-frequency antenna unit. Here, the parameters and/or information that can be input by means of the touch display directly inside the examination area 27 during the preparation of the patient 18, can be a selection of an examination region and/or of a patient position and/or of further patient-specific parameters. In addition, such parameters and/or information can also be a confirmation of the implementation of a step in the procedure that is displayed by means of the touch display 35 to the medical operator 34.

Suggestions and/or instructions for various medical imaging examinations that can be retrieved by the medical operator 34 when implementing the respective medical imaging examination by means of the touch display 35 are preferably stored in the memory unit of the system control computer 28, in particular of the operation control processor 36. In this way, the individual medical imaging examinations can be very heavily automated and also carried out in a simple and safe manner even by an inexperienced medical operator 34 by means of the user interface 33.

Control of the user interface 33 is achieved by the operation control processor 36. For this purpose, an exchange 42 ensues between the user interface 33 and the operation control processor 36 of control data that include both data to control the user interface 33 and also data relating to an input by the medical operator 34, which are transmitted to the operation control processor 36. Moreover, the system control computer 28 also generates control commands for the control of the magnetic resonance apparatus 12, an exchange 43 of these control data ensuing between the system control computer 28 and the magnetic resonance apparatus 12 to control the magnetic resonance apparatus 12. The control data to control the magnetic resonance apparatus 12 can, moreover, also be dependent on the inputs, in particular on the control parameters that have been input on the touch display 35. Here, too, an exchange 43 of further data, such as status information and/or error reports and so on can also ensue between the magnetic resonance apparatus 12 and the system control computer 28.

Furthermore, the method makes provision for supervision of the medical imaging examination to take place on an operating console 30. The supervision can be carried out by a further user, by for example, a physician familiar with the medical imaging examination. The operating console may include the operating console 30 of the medical imaging system 10, which is arranged inside the control area 29. In addition, the operating console 30 can additionally be arranged outside the control area 29, such that the further user has external access to the medical imaging system 10. The external access to the medical imaging system 10 can be achieved through the internet and/or an intranet.

Due to the fact that the medical operator 34 stays inside the examination area 27 during the medical imaging examination, it is advantageous if the medical imaging apparatus 12 can be operated in a particularly noise-free manner. This is an advantage in particular when the medical imaging apparatus 12 is configured as a magnetic resonance apparatus, since operation of a magnetic resonance apparatus generates loud noises. Therefore, the magnetic resonance apparatus, in particular the housing 21 of the scanner 13, is insulated against propagation of sound waves and/or vibrations. For this purpose, the housing 21 has an insulating layer that is not shown in greater detail, which allows the medical operator 34 to stay inside the examination area 27 during a magnetic resonance examination.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for operating a medical imaging system comprising a medical data acquisition seamier configured to interact with a patient, and a control computer configured to operate the data acquisition scanner, said data acquisition scanner being situated in an examination area and said control computer being situated in a control area that is physically separated from said examination area, said method comprising:
providing a user interface inside said examination area; and
during said medical imaging examination, implementing at least one of presentation of information or suggestions, and entry of operating parameters or information, via said user interface at a location in said examination area.

2. A method as claimed in claim 1 comprising implementing said presentation or said entry via said user interface during a preparation of said medical imaging examination.

3. A method as claimed in claim 1 comprising providing a memory in communication with said control computer, and storing said presentation information or suggestions, or said entry of parameters or information, in said memory.

4. A method as claimed in claim 1 comprising providing an operating processor in said control computer, and controlling said user interface via said operating processor.

5. A method as claimed in claim 1 comprising providing an operating console, in communication with said control computer, in said control area, and supervising said medical imaging examination from said operating console.

6. A medical imaging system comprising:
a medical data acquisition scanner configured to interact with a patient;
a control computer configured to operate the data acquisition scanner, said data acquisition scanner being situated in an examination area and said control computer being situated in a control area that is physically separated from said examination area;
a user interface inside said examination area; and
said user interface at a location in said examination area being configured to implement, during said medical imaging examination, at least one of presentation of information or suggestions, and entry of operating parameters or information.

7. A medical imaging system as claimed in claim 6 wherein said user interface comprises a touch display.

8. A medical imaging system as claimed in claim 6 wherein said user interface is fixedly mounted on said data acquisition scanner.

9. A medical imaging system as claimed in claim 6 wherein said user interface is removably mounted on said data acquisition scanner.

10. A medical imaging system as claimed in claim 6 wherein said control computer comprises an operating processor, said operating processor being configured to control said user interface.

11. A medical imaging system as claimed in claim 6 comprising an operating console in communication with said control computer in said control area.
